(19) United States
(12) Patent Application Publication
(10) Pub. No.: US 2011/0315147 A1
(43) Pub. Date: Dec. 29, 2011

(54) TRACHEAL TUBE WITH MULTIFUNCTIONAL LUMEN

(75) Inventors: Lockett E. Wood, Lyons, CO (US); Dawn Bitz, Boulder, CO (US); Dana Deardorff, Lafayette, CO (US)

(73) Assignee: Nellcor Puritan Bennett LLC, Boulder, CO (US)

(21) Appl. No.: 12/825,912

(22) Filed: Jun. 29, 2010

(51) Int. Cl. A61M 16/04

(52) U.S. Cl. 128/207.15

(57) ABSTRACT

Various embodiments of a tracheal tube having a multifunctional lumen disposed in a tracheal tube wall are provided. Provided tracheal tubes include a ventilation lumen, through which bidirectional airflow may be established to and from a patient during operation, as well as a multifunctional lumen adapted to receive a variety of interchangeable medical devices. Embodiments of the provided tracheal tubes may provide for insertion or removal of a medical device from the multifunctional lumen without extubation of the tracheal tube from a patient.

### BACKGROUND

The present disclosure relates generally to medical devices and, more particularly, to airway devices, such as tracheal tubes.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Tracheal tubes are often placed in the airway of a patient in medical situations that necessitate protection of the airway from possible obstruction or occlusion. For instance, tracheal tubes may be used in emergency situations, such as when a patient experiences cardiac or respiratory arrest. Oftentimes, when a patient experiences cardiac arrest, ventilation with a traditional mask may be unfeasible and/or ineffective, thus necessitating use of a tracheal tube. For further example, when a patient experiences a coma or areflexia, the ability of the patient to breathe without assistance may be compromised, and a tracheal tube may be inserted for ventilation purposes.

Such tracheal tubes are often coupled to an air source, such as a ventilator, to provide the patient with a source of fresh air. Accordingly, traditional tracheal tubes include a main ventilation lumen adapted to allow airflow to and from the patient during inspiration and expiration, respectively. However, it may be desirable for additional functionalities to be provided by the tracheal tube. For example, a variety of tracheal tubes including a suction lumen opening in a port proximate an inflatable balloon on the tube are known in the art. These suction lumens enable continuous or intermittent suctioning of secretions that traverse the epiglottis and vocal chords and accumulate above the cuff or on the tube. Because such secretions often contain infectious microorganisms, tubes having such suction lumens may be desirable in certain clinical circumstances.

Tracheal tubes that provide additional functionalities, such as secretion suctioning or tubes that allow for introduction of a medicament, are presently only offered as specialty devices. That is, before the decision to intubate a patient is made, the clinician must decide on the preferred tracheal tube—either between a basic tube that simply permits ventilation or a specialty tube that offers an additional single functionality (e.g., suction of secretions). Most often for reasons of cost, familiarity, or otherwise, the clinician simply chooses a basic tracheal tube to establish an airway and begin ventilating a patient. Later, however, it may be clinically desirable to have a specialty tube that would permit some additional functionality beyond simple ventilation. Because many patients remain intubated for long periods of time and may be moved, for example between an emergency room or operating room to the intensive care unit, or because complications are often unforeseen, the decision of which tracheal tube to use initially may be complicated and prone to risk. Moreover, once the initial tube is placed, it may be clinically undesirable and potentially harmful to remove that tracheal tube from the patient for the purpose of placing a new tracheal tube with an added functionality. Placing a tube with additional functionalities initially, on the other hand, carries its own potential drawbacks because such tubes are often substantially more expensive, or may be unfamiliar or complicated. Depending on the development of a particular patient's clinical situation, the added expense or other factors may ultimately prove to be unjustified. Accordingly, there remains a need for tracheal tubes, systems, and methods that permit a more cost effective, “first-in” tube that enables “on the fly” modification to satisfy a particular clinical need at the time, and only when a particular functionality or combination of functionalities is clinically desirable. As described below, such additional functions may include drug delivery, pressure monitoring, physiological or chemical monitoring, visualization, and the like.

### BRIEF DESCRIPTION OF THE DRAWINGS

Advantages of the disclosed techniques may become apparent upon reading the following detailed description and upon reference to the drawings in which:

FIG. 1 is an elevational view of an exemplary tracheal tube including a multifunctional lumen disposed therein;

FIG. 2 is a flow chart illustrating an exemplary method of operating the tracheal tube of FIG. 1;

FIG. 3 illustrates an exemplary tracheal tube having an increased wall thickness sized to accommodate the multifunctional lumen of FIG. 1;

FIG. 4 illustrates an exemplary tracheal tube including a varying wall thickness sized to accommodate the multifunctional lumen of FIG. 1;

FIG. 5 illustrates an exemplary tracheal tube including an exterior protrusion sized and shaped to accommodate the multifunctional lumen of FIG. 1;

FIG. 6 illustrates an exemplary tracheal tube including an interior protrusion sized and shaped to accommodate the multifunctional lumen of FIG. 1;

FIG. 7 illustrates an exemplary tracheal tube including an exterior track sized and shaped to accommodate the multifunctional lumen of FIG. 1;

FIG. 8 illustrates an exemplary tracheal tube including an interior track sized and shaped to accommodate the multifunctional lumen of FIG. 1;

FIG. 9 illustrates an exemplary tracheal tube including multiple multifunctional lumens disposed in a tracheal tube wall;

FIG. 10 illustrates an exemplary tracheal tube including multiple multifunctional lumens disposed in a tracheal tube wall and sealed with plugging devices; and

FIG. 11 illustrates an exemplary tracheal tube including multiple multifunctional lumens disposed in a tracheal tube wall and a connector with self-sealing ports.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As described in detail below, embodiments of an endotracheal tube (ETT) are provided having a multifunctional lumen disposed in a tube wall. That is, the provided tracheal tubes include a ventilation lumen, through which bidirectional airflow may be established to and from a patient during operation, as well as a multifunctional lumen adapted to receive a variety of interchangeable medical devices. As such, the multifunctional lumen may reduce or eliminate the need for a medical practitioner to determine the function of the multifunctional lumen prior to intubation. After intubation of a patient with the tracheal tube, one of a variety of specialty devices may be chosen by the user for insertion into the multifunctional lumen. Still further, embodiments of the disclosed tracheal tube may provide for removal of the chosen medical device without extubation of the tracheal tube from the patient. As such, during intubation, a variety of devices may be inserted and removed from one or more multifunctional lumens disposed in a sidewall of the tracheal tube, and such devices may be determined after patient intubation with the multifunctional tracheal tube.

The foregoing features of the multifunctional tracheal tubes described herein may offer distinct advantages over traditional tracheal tubes. For instance, traditional tracheal tubes may be specially designed to provide a specific functionality or may be designed solely for patient ventilation without provisions for additional functionalities. As such, traditional tracheal tubes may require a specialty tracheal tube to be chosen prior to intubation if functionalities beyond basic ventilation are desired. Unfortunately, such specialty tracheal tubes may be of high monetary cost, and it is difficult to predict which functionalities will be needed during long term patient care. Embodiments of the multifunctional tracheal tubes disclosed herein, however, may offer distinct advantages over such tubes since the disclosed tracheal tubes may enable functionalities to be chosen after patient intubation. Furthermore, since the functionalities need not be chosen before patient intubation, the presently disclosed tracheal tubes may offer a relatively low cost alternative to high cost specialty tracheal tubes, because the disclosed embodiments may be further adapted as desired after intubation. That is, embodiments of the disclosed tracheal tubes may include the multifunctional lumen, which allows for further adaptations after intubation, but may only provide ventilation capabilities upon initial intubation. As such, the multifunctional tracheal tubes may be initially low cost ventilation devices that are capable of receiving additional devices if desired after intubation. Still further, traditional tracheal tubes may involve insertion of one or more devices such that the established breathing circuit of the patient may be disrupted. Embodiments of the presently disclosed tracheal tubes, however, may provide the multifunctional lumen, which facilitates insertion of additional devices without interruption of the patient's breathing cycle (i.e., through the multifunctional lumen)

The ETT may be disposable rather than reusable, capable of conveying gas to and from the patient, and capable of facilitating insertion and removal of a variety of specialty medical devices into the patient without necessitating extubation of the patient. As such, the devices and techniques provided herein may enable the ability to intubate a patient with a single multifunctional tracheal tube that is adapted to receive any of a variety of desirable medical devices. Accordingly, during operation, the multifunctional lumen may be adapted to receive a drug delivery device, a pressure monitoring device, an imaging or visualization device, a position monitoring device, or any other specialty medical device.

It should be noted that the provided tracheal tubes and methods of operating the tracheal tubes may be used in conjunction with auxiliary devices, such as airway accessories, ventilators, humidifiers, and so forth, which may cooperate with the tracheal tubes to maintain airflow to and from the lungs of the patient. For instance, the tracheal tubes may be placed in the trachea and coupled to a ventilator to protect the airway from possible obstruction or occlusion in emergency situations, such as when a patient experiences cardiac or respiratory arrest. That is, embodiments of the presently disclosed tracheal tubes may be utilized when ventilation with a traditional mask may be unfeasible and/or ineffective, thus necessitating use of a tracheal tube. For example, when a patient experiences a coma or areflexia, the ability of the patient to breathe without assistance may be compromised, and a tracheal tube may be inserted for ventilation purposes. Still further, such tracheal tubes may be coupled to a connector that is adapted to interface with the tracheal tube to facilitate insertion of one or more medical devices into the multifunctional lumen.

Furthermore, although the embodiments of the present invention illustrated and described herein are discussed in the context of endotracheal tubes, it should be noted that presently contemplated embodiments may include a multifunctional lumen disposed in a wall of any of a variety of suitable airway devices. For example, the multifunctional lumen may be associated with a tracheostomy tube, a Broncho-Cath™ tube, a specialty tube, or any other airway device with a main ventilation lumen defined by a tubular wall. Indeed, any device with a ventilation lumen designed for use in an airway of a patient may include a multifunctional lumen disposed therein to facilitate the insertion of a variety of medical devices. Furthermore, as used herein, the term “tracheal tube” may include an endotracheal tube, a tracheostomy tube, a Broncho-Cath™ tube, a specialty tube, or any other suitable airway device.

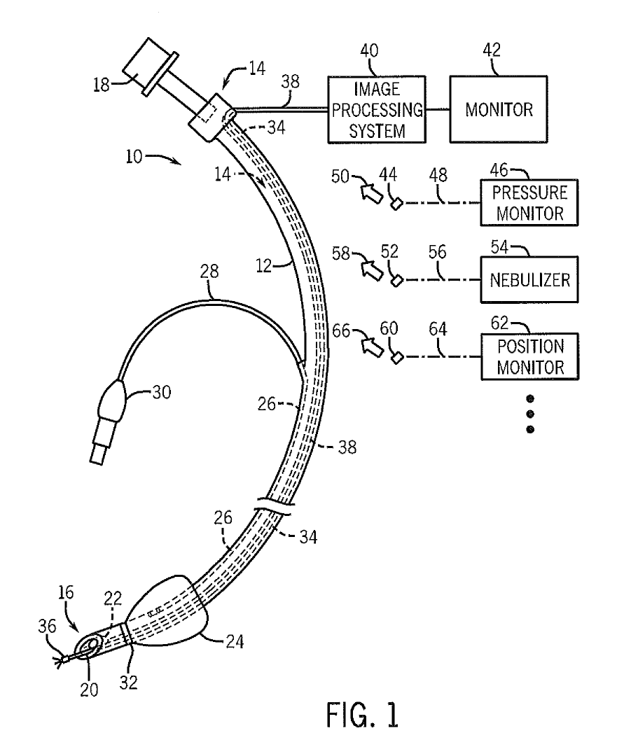

Turning now to the drawings, FIG. 1 is an elevational view of an exemplary tracheal tube 10 in accordance with aspects of the present disclosure. The tracheal tube 10 includes a central tubular body 12 with proximal and distal ends 14 and 16, respectively. In the illustrated embodiment, the proximal end 14 is outfitted with a connector 18 that may be attached to a mechanical ventilator during operation. The distal end 16 terminates in an opening 20 and may be placed in a patient's trachea during operation to maintain airflow to and from the patient's lungs. A Murphy's eye 22 may be located on the tubular body 12 opposite the opening 20 to prevent airway occlusion when the tube assembly 10 is improperly placed within the patient's trachea.

As illustrated, a cuff 24 that may be inflated to seal against the walls of a body cavity (e.g., a trachea) may be attached to the distal end 16 of the tubular body 12. The cuff 24 may be inflated via an inflation lumen 26 terminating in an inflation tube 28 connected to a fixture 30 located at the proximal end 14 of the tubular body 12. A shoulder 32 of the cuff 24 secures the cuff 24 to the tubular body 12. In some embodiments, the shoulder 32 may be folded up inside a lower end of the cuff 24 (not shown). Furthermore, the tubular body 12 and the cuff 24 may be formed from materials having desirable mechanical properties (e.g., puncture resistance, pin hole resistance, tensile strength, and so forth) and desirable chemical properties (e.g., biocompatibility). In one embodiment, the walls of the cuff 24 may be made of a polyurethane (e.g., Dow Pellethane® 2363-80A) having suitable mechanical and chemical properties. In other embodiments, the walls of the cuff 24 may be made of a suitable polyvinyl chloride (PVC). In certain embodiments, the cuff 24 may be generally sized and shaped as a high volume, low pressure cuff that may be designed to be inflated to pressures between about 15 cm H₂O and 30 cm H₂O.

As illustrated, the tubular body 12 also includes a multifunctional lumen 34 that extends from a location on the tracheal tube 10 positioned outside the body when in use to a location below the Murphy's Eye 22 opening into the patient's trachea. In the illustrated embodiment, a camera 36 has been inserted through the multifunctional lumen 34. As such, a cable 38 terminating in the camera 36 is disposed in the lumen 34. The cable 38 couples the camera 36 to an image processing system 40, which is further coupled to a monitor 42. During operation, the camera 36 may be configured to acquire data representative of one or more images from within the patient's trachea and to transmit the acquired data to the image processing system 40 via cable 38. The image processing system 40 is adapted to receive the acquired data and to process such data to an output suitable for viewing on the monitor 42. In certain embodiments, processing system 40 and monitor 42 may be separate components or may be unitary.

It should be noted that the camera 36 may be utilized in the multifunctional lumen 34 during a variety of suitable instances during patient care. For example, the camera 36 may be utilized while the patient is being intubated to provide placement and positioning guidance. For further example, the camera 36 may be inserted after intubation while the patient is being ventilated to image one or more areas of the trachea. Still further, the camera 36 may be deployed during extubation and used to guide removal of the tracheal tube from the patient's trachea.

It should be noted that although the camera 36 is illustrated in the lumen 34 in the shown embodiment, the multifunctional lumen 34 is adapted to receive a variety of medical devices. For example, the multifunctional lumen 34 may be adapted to receive a pressure sensor 44 coupled to an external pressure monitor 46 via cable 48, as indicated by arrow 50. The multifunctional lumen 34 may also be adapted to receive a drug delivery device 52 coupled to an external nebulizer 54 via cable 56, as indicated by arrow 58. Still further, the lumen 34 may be configured to receive a position sensor 60 coupled to a position monitor 62 via cable 64, as indicated by arrow 66. Indeed, the multifunctional lumen 34 may be adapted to receive any specialty medical device that may be coupled to a specialty externally located device. Specialty medical devices may include but are not limited to a temperature sensor, a heart rate monitor, an electrode for measuring transthoracic impedance, an analytic sensor (e.g. a pH sensor or a gas sensor, such as an oxygen sensor or a carbon dioxide sensor), a humidity sensor, and a disease sensor (e.g., a fiber optic cable terminating in a sensor including one or more antibodies).

Additionally, it should be noted that although the devices illustrated in FIG. 1 are coupled to cables 48, 56, and 64, in additional embodiments, the devices inserted through the multifunctional lumen 34 may be adapted to communicate wirelessly without the illustrated cables. For example, the camera 36 may be configured to communicate data to the image processing system 40 wirelessly. Nevertheless, in embodiments in which the cables 48, 56, and 64 are utilized, such cables may be of sufficient rigidity to traverse lengthwise through the lumen 34 when pushed by a medical technician. That is, the cables 48, 56, and 64 may be flexible enough to take on the shape of the deployed tracheal tube but rigid enough to be inserted when a push force is externally applied.

In one embodiment, for example, the multifunctional lumen 34 may be sized and shaped such that the lumen 34 may interchangeably accommodate the illustrated medical devices. In such an embodiment, the multifunctional lumen 34 may be sized to accommodate the camera 36 during insertion through the lumen 34 and during removal from the lumen 34. Accordingly, in one embodiment, the multifunctional lumen 34 may have a diameter between approximately 1.5 mm and approximately 3 mm. For further example, the lumen 34 may have a diameter between approximately 2 mm and approximately 3 mm. Still further, the lumen 34 may have a diameter between approximately 2.5 mm and approximately 3 mm. For example, the multifunctional lumen 34 may have a diameter of up to approximately 1 mm, up to approximately 2 mm, or up to approximately 3 mm, such that the lumen 34 may accommodate insertion and removal of devices (e.g., cameras) that may not be suitable for insertion into lumens of traditional tracheal tubes.

Still further, additional modifications may be made to the multifunctional lumen 34 to facilitate the insertion and removal of the desired medical devices. For example, the multifunctional lumen 34 may be coated with one or more desirable layers, such as a lubricant, which allows the desired medical devices to slide within the lumen 34. Additionally, in the illustrated embodiment, a single multifunctional lumen 34 is shown. However, in further embodiments, a plurality of multifunctional lumens may be located radially around the tubular body 12 such that medical devices may be inserted in a variety of radial positions in the airway of an intubated patient.

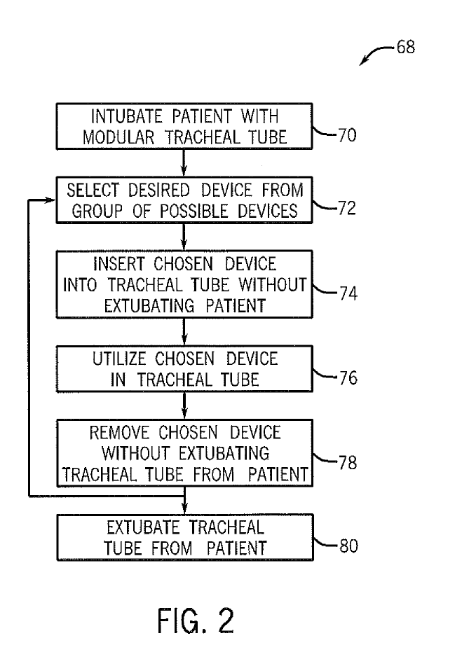

FIG. 2 is a flow chart illustrating an exemplary method 68 that may be employed in conjunction with the tracheal tube 10 of FIG. 1. The method 68 includes the step of intubating the patient with the modular tracheal tube with the multifunctional lumen (block 70). After intubation, a desired device is selected from a group of possible devices for insertion into the patient's trachea via the multifunctional lumen (block 72). That is, post-intubation, a user may select a device to insert into the trachea of the patient via the multifunctional lumen without the need to remove the tracheal tube and/or modify the tracheal tube. As such, the method 68 further includes inserting the chosen device into the tracheal tube without extubating the patient (block 74). Additionally, the chosen device may be inserted into the tracheal tube without interruption of the breathing circuit established between the patient and the ventilation device. That is, the patient need not be extubated, nor does the ventilator or other breathing source need to be disconnected or stopped, such that the breathing circuit may be maintained for insertion of the chosen device. Thus, ventilation or assisted breathing may continue uninterrupted during insertion of the chosen device, or any change in devices. The user may then utilize the selected device in the tracheal tube and/or in the patient's trachea (block 76). For example, the user may acquire one or more digital images with the camera, deliver a drug to the patient, measure a pressure, and so forth while the patient is intubated.

It should be noted that the chosen devices may be utilized in the multifunctional lumen at a variety of suitable instances during patient care. For example, in one embodiment, the chosen device may be inserted, utilized, and removed during intubation. However, in further embodiments, the chosen device may be inserted prior to intubation or during extubation. Still further, the chosen device need not be removed from the multifunctional lumen prior to patient extubation. For example, a camera may remain in the multifunctional lumen during extubation to guide the process of removing the tracheal tube from the trachea.

The selected device may then be removed from the multifunctional lumen without extubating the tracheal tube from the patient (block 78). If desired, the user may then select another device from the group of possible devices (block 72) to insert into the patient's trachea. Again, the selected device may be inserted into the lumen, utilized while located in the lumen, and extubated from the tracheal tube without removal of the tracheal tube from the patient (blocks 74, 76, and 78). Such steps may be repeated by the user as desired until it is no longer necessary to insert any additional devices in the multifunctional lumen. That is, a first device may be chosen, inserted into the lumen, utilized in the lumen, and removed from the lumen without extubating the patient. Subsequently, a second device may be chosen, inserted into the same lumen or a different multifunctional lumen, utilized in the lumen, and removed from the lumen without extubating the patient. Indeed, any number of devices may be sequentially inserted into and removed from the same lumen during intubation. Finally, the tracheal tube may be removed from the patient (block 80), such as when the patient has recovered and is able to breathe without assistance.

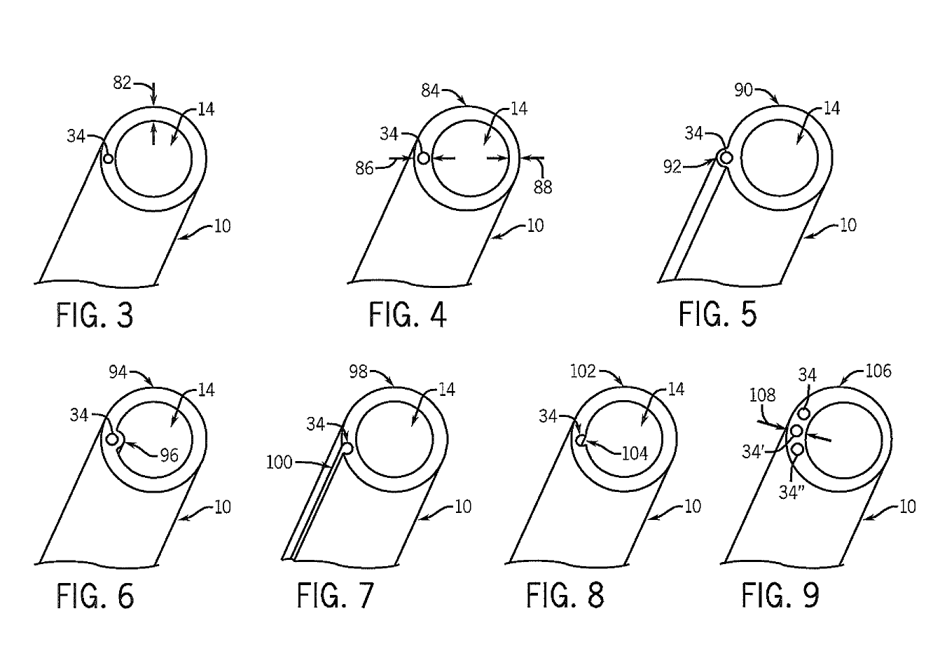

FIG. 3 illustrates an exemplary tracheal tube 10 including an increased wall thickness 82 appropriately sized and shaped to accommodate the multifunctional lumen 34. That is, as compared to traditional tracheal tubes, which have a wall thickness equal to approximately 1 mm, embodiments of the presently disclosed tracheal tube 10 may have an increased wall thickness 82 greater than the diameter of the multifunctional lumen (e.g., a wall thickness of 2.5 mm for a 1.5 mm lumen, and so forth for lumens of other sizes). Such an increased thickness in the wall of the tracheal tube may enable the multifunctional lumen to be appropriately sized to facilitate the insertion and removal of a plurality of medical devices, such as cameras, the size of which may prevent such devices from being inserted into traditional lumens (e.g., inflation lumens, suction lumens, etc.). Additionally, the increased thickness 82 of the wall may be limited such that the thickness is sized large enough to accommodate the desired medical devices but no larger than is necessary such that the stiffness of the tracheal tube wall is limited.

FIG. 4 illustrates an alternate exemplary embodiment of the tracheal tube 10 including the multifunctional lumen 34 located in a tracheal tube wall 84 with a variable thickness. That is, a first thickness 86 of a first side of the tracheal tube wall 84 is substantially larger than a second thickness 88 of a second side of the tracheal tube wall. The tracheal tube wall 84 may be sized in such a way to accommodate the multifunctional lumen 34 in the first side of the wall with the first thickness 86. The foregoing feature may offer distinct advantages over designs in which the tube wall 84 is of uniform thickness around the entire circumference since the flexibility of the variable thickness tracheal tube may be substantially greater than the flexibility of a tube with a uniformly increased thickness. Furthermore, such a design may limit the reduction in volume of the ventilation lumen 14 as compared to tracheal tubes without the multifunctional lumen 34.

It should be noted that the area of increased wall thickness 84 may be advantageously placed around the circumference of the tracheal tube. For example, the increased wall thickness 84 may be placed such that the Magill curvature of the tracheal tube is maintained during operation. Still further, more than one area of increased thickness may be provided in the tracheal tube. For example, a first thickened area and a second thickened area may be provided substantially opposite one another such that the ventilation lumen is substantially oval shaped during operation.

FIG. 5 illustrates an exemplary embodiment of the tracheal tube 10 including the multifunctional lumen 34 disposed in a modified tracheal tube wall 90. In this embodiment, the modified tracheal tube wall 90 includes a protrusion 92, which enlarges the tracheal tube wall 90 selectively at the location of the multifunctional lumen 34. That is, the tube wall 90 is modified only in the location of the lumen 34 to accommodate the size of the lumen 34 via the protrusion 92. In this embodiment, the protrusion 92 is positioned on an outside of the tubular wall, such that the size and shape of the ventilation lumen 14 remains substantially the same as compared to traditional tracheal tubes. The protrusion 92 may have the effect of reducing or eliminating an increase in stiffness of the tracheal tube 10 as compared to traditional tracheal tube that do not include the multifunctional lumen 34.

FIG. 6 illustrates an exemplary embodiment of the tracheal tube 10 including the multifunctional lumen 34 disposed in a modified tracheal tube wall 94. In this embodiment, the modified tracheal tube wall 94 includes a protrusion 96, which enlarges the tracheal tube wall 90 selectively at the location of the multifunctional lumen 34 as in the embodiment of FIG. 5. However, in this embodiment, the protrusion 96 is positioned on the inside of the tubular wall 94, such that the protrusion 96 extends within the ventilation lumen 14, thereby slightly reducing the volume of the ventilation lumen and altering the shape of the inner wall of the tracheal tube 10. However, as before, the tube wall 94 is modified only in the location of the lumen 34 to accommodate the size of the lumen 34 via the protrusion 96.

FIG. 7 illustrates an exemplary embodiment of the tracheal tube 10 including the multifunctional lumen 34 positioned in a tracheal tube wall 98 and designed as an exterior track 100. In this embodiment, the exterior track 100 in the lumen 34 is sized and shaped to interchangeably receive one or more medical devices. Furthermore, the exterior track 100 is disposed in an exterior of the tracheal tube wall 98 such that the size and shape of the ventilation lumen 14 remains substantially unchanged as compared to traditional tracheal tubes that do not include a multifunctional lumen 34. During operation, a medical device may be inserted into the patient's trachea via the exterior track 100 and removed when desired without extubating the patient.

FIG. 8 illustrates an exemplary embodiment of the tracheal tube 10 including the multifunctional lumen 34 positioned in a tracheal tube wall 102 and designed as an interior track 104. In this embodiment, the interior track 104 in the lumen 34 is sized and shaped to interchangeably receive one or more medical devices. Furthermore, the interior track 104 is disposed in an interior of the tracheal tube wall 102 such that the shape of the ventilation lumen 14 is altered to accommodate the track 104 as compared to traditional tracheal tubes that do not include a multifunctional lumen 34. As before, during operation, a medical device may be inserted into the patient's trachea via the track 104 and removed when desired without extubating the patient.

It should be noted that the tracks 100 and 104 of FIGS. 7 and 8 may include additional modifications not shown. For example, the tracks 100 and 104 may include one or more rails adapted to facilitate the insertion of medical devices into the patient's trachea. The one or more rails may also be configured to maintain one or more cables associated with the inserted medical devices in the tracks 100 and 104 such that the cables may be maintained in the multifunctional lumen 34 and substantially prohibited from entering the ventilation lumen 14. Still further, the tracks may be in a more or less open configuration than is illustrated. That is, the track may be fully enclosed, partially enclosed, or substantially open.

Still further, in the embodiments of FIGS. 7 and 8, additional modifications may be provided in embodiments in which the tracheal tube 10 includes a cuff. For example, in one such embodiment, the tracks 100 and 104 may be adapted to terminate above the cuff so as not to interfere with the sealing properties of the cuff. In another embodiment, the tracks may be adapted to be routed through a closed lumen in the area of the cuff such that the tracks terminate below the cuff without interfering with the seal between the cuff and the patient's trachea. Indeed, any suitable modifications may be made to the tracks in embodiments which employ cuffed tracheal tubes to ensure that the sealing properties of the cuff are not impacted by the presence of the tracks.

FIG. 9 illustrates an exemplary embodiment of the tracheal tube 10 including a plurality of multifunctional lumens 34, 34′, and 34″ disposed in a wall 106 of the tracheal tube 10. As such, this embodiment may include more than one multifunctional lumen 34 disposed in the same tracheal tube wall 106. In such embodiments, multiple devices, which need not be determined prior to intubation, may be simultaneously inserted and removed from the tracheal tube 10 as desired by the operator. That is, the user may utilize multiple devices at the same time by inserting such devices in the multiple lumens 34, 34′, and 34″.

It should be noted that the tracheal tube 10 of FIG. 9 may be further adapted to include modifications not shown. For example, a thickness 108 of the wall 106 in the location of the multifunctional lumens 34, 34′, and 34″ may be increased as compared to the thickness of the wall in the positions where the multifunctional lumens are not located. For further example, although three lumens are illustrated, additional lumens may be included in the wall 106. Still further, the lumens may be positioned in predetermined positions such that the inserted medical devices are deployed in advantageous positions within the patient's trachea (e.g., a lumen positioned in an area without mucus accumulation to facilitate acquisition of imaging data). In some embodiments, the lumens may terminate at different locations along the tracheal tube body (e.g., above the cuff, below the cuff, and so forth) determined by the purpose of the specialty device the lumens are each respectively configured to receive. Indeed, any number of multifunctional lumens may be arranged in any suitable manner in the wall (e.g., radially spaced about the circumference of the wall, selectively positioned in discrete locations, etc.). For example, in one embodiment, the tracheal tube may include four lumens equidistantly disposed radially about the circumference of the tracheal tube, such that each lumen is disposed 90 degrees from each adjacent lumen.

Still further, embodiments of the presently disclosed tracheal tubes that include multiple multifunctional lumens may be further adapted such that each multifunctional lumen is designed to accommodate a particular device. For example, a first lumen may be sized to accommodate a first medical device (e.g., a camera), and a second lumen may be sized to accommodate a second medical device (e.g., a pressure sensor). For further example, a first lumen and a second lumen may be sized to accommodate similar first and second devices (e.g., a first imaging device and a second imaging device). In such embodiments, each lumen may be marked such that an operator may identify which lumen is adapted to receive which device. For example, the first lumen may be marked with a “C” and the second lumen may be marked with a “PS” to indicate to the operator which lumen is adapted for which device. Still further, other embodiments may generically mark the multifunctional lumens (e.g., sequential numbering system) such that after a device has been inserted, the operator may easily identify which lumen contains which device as the patient moves or is repositioned. For further example, the lumens may each be marked with a distinct color (e.g., the first lumen may be marked red and the second lumen may be marked blue) or a distinct structure (e.g., the first lumen may be marked with one raised dot and the second lumen may be marked with two raised dots). Indeed, such markings may be utilized to indicate a variety of suitable information to a user, such as which device is located in which lumen, a radial position of each lumen around the circumference of the tracheal tube, and so forth.

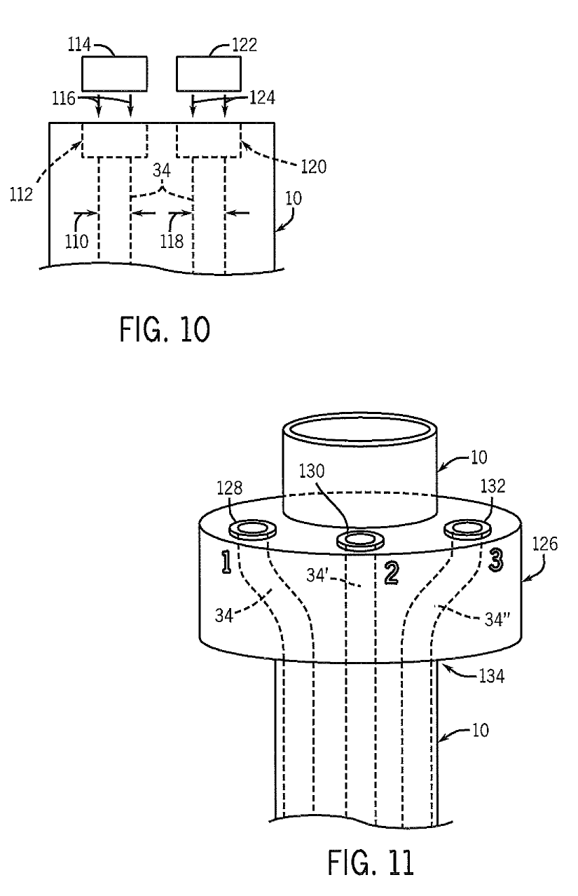

FIG. 10 illustrates the tracheal tube 10 with multifunctional lumens 34 and 34′ adapted to be sealed when not in use. That is, the first lumen 34 of a first width 110 may include an enlarged portion 112 that is configured to receive a plugging device 114, as indicated by arrows 116 when not in use. Similarly, the second lumen 34′ of a second width 118 may include an enlarged portion 120 that is configured to receive a plugging device 122, as indicated by arrows 124, when not in use. As such, the plugging devices 114 and 122 may be adapted to be selectively inserted and removed from the enlarged portions 112 and 120 by an operator when medical devices are inserted or removed from the lumens 34 and 34′. As before, the first width 110 may be equal to or different from the second width 118 depending on the given application.

FIG. 11 illustrates an embodiment of the tracheal tube 10 outfitted with a connector 126 adapted to facilitate insertion and removal of one or more medical devices from the tracheal tube 10. As such, the tracheal tube 10 includes multifunctional lumens 34, 34′, 34″ disposed throughout both the wall of the tracheal tube 10 as well as the connector 126. The first multifunctional lumen 34 terminates in a first self-sealing port 128, the second multifunctional lumen 34′ terminates in a second self-sealing port 130, and the third multifunctional lumen 34″ terminates in a third self-sealing port 132. The self-sealing ports 128, 130, and 132 may be adapted to form a seal between the exterior environment and an interior of the connector and the lumens 34, 34′, and 34″ when medical devices are not disposed in the lumens.

The self-sealing ports may be further adapted to allow for insertion of a medical device without the removal of a plugging device and to encircle around the inserted device to create a seal between the exterior environment and the interior of the lumen during operation. Upon removal of the inserted device, the self-sealing ports may be configured to reseal, thereby reestablishing the seal between the lumen and the exterior environment upon removal of the device. As such, the self-sealing ports may reduce or eliminate the risk of undesirable contaminants from the external environment contaminating the lumens and entering the patient. It should be noted that although the self-sealing ports are disposed in the connector 126 in the illustrated embodiment, in further embodiments, the self-sealing ports may be located at an interface 134 between the connector 126 and the tracheal tube 10 or both at the interface 134 and at the top of the connector 126.

Furthermore, it should be noted that a variety of other suitable self sealing methods and devices may be employed in conjunction with the multifunctional lumens. For example, any suitable valve arrangement adapted to allow insertion and removal of the desired medical device while preventing contamination from elements of the surrounding environment may be employed. For further example, a variety of chemical and/or physical substances may be employed at the proximal end of the multifunctional lumens to seal around an inserted medical device while inserted and to reseal the proximal end of the multifunctional lumen when the medical device is removed.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A multifunctional tracheal tube system, comprising:
a tubular body having an open distal end;
a ventilation lumen disposed in the tubular body and configured to facilitate airflow to and from a patient; and
a multifunctional lumen disposed in a wall of the tubular body and configured to interchangeably receive at least two different specialty devices.

2. The multifunctional tracheal tube system of claim 1, wherein the specialty devices comprise an imaging device, a drug delivery device, a position sensor, a chemical sensor, and a pressure sensor.

3. The multifunctional tracheal tube system of claim 1, further comprising a second multifunctional lumen disposed in a wall of the tubular body and configured to interchangeably receive at least two specialty devices.

4. The multifunctional tracheal tube system of claim 3, wherein the at least two specialty devices comprise an imaging device, a drug delivery device, a position sensor, a chemical sensor, and a pressure sensor.

5. The multifunctional tracheal tube system of claim 3, wherein the tubular body comprises a first marking associated with the multifunctional lumen and a second marking associated with the second multifunctional lumen.

6. The multifunctional tracheal tube system of claim 1, wherein the multifunctional lumen terminates in an enlarged portion configured to receive a plugging device adapted to seal an interior of the multifunctional lumen from a surrounding environment.

7. The multifunctional tracheal tube system of claim 1, wherein a thickness of the wall of the tubular body is increased in a region of the wall surrounding the multifunctional lumen.

8. The multifunctional tracheal tube system of claim 1, wherein the multifunctional lumen is disposed in a protrusion extending outward from a surface of the wall of the tubular body.

9. The multifunctional tracheal tube system of claim 1, wherein the multifunctional lumen comprises a track disposed in the wall of the tracheal tube body.

10. The multifunctional tracheal tube system of claim 1, wherein the multifunctional lumen terminates in a self-sealing port configured to encircle the received device when inserted into the multifunctional lumen and to seal upon removal of the received device to maintain a separation between an interior of the multifunctional lumen and a surrounding environment.

11. A multifunctional tracheal tube system, comprising:
a tracheal tube comprising a tubular body, a ventilation lumen disposed in the tubular body and configured to facilitate airflow to and from a patient, and a multifunctional lumen disposed in a wall of the tubular body;
at least two specialty devices; and
wherein the multifunctional lumen is configured to interchangeably receive the at least two devices.

12. The multifunctional tracheal tube system of claim 11, wherein the multifunctional lumen terminates in a self-sealing port configured to encircle the received device when inserted into the multifunctional lumen and to seal upon removal of the received device to maintain a separation between an interior of the multifunctional lumen and a surrounding environment.

13. The multifunctional tracheal tube system of claim 11, wherein the tracheal tube comprises a second multifunctional lumen disposed in the wall of the tubular body, and wherein the multifunctional lumen is sized to receive a first of the at least two medical devices and the second multifunctional lumen is sized to receive a second of the at least two medical devices.

14. The multifunctional tracheal tube system of claim 13, wherein the tracheal tube includes a first marking indicating the multifunctional lumen and a second marking indicating the second multifunctional lumen.

15. The multifunctional tracheal tube system of claim 11, wherein a diameter of the multifunctional lumen is equal to approximately 3 mm.

16. A multifunctional tracheal tube system, comprising:
a tubular body having an open distal end;
a ventilation lumen disposed in the tubular body and configured to facilitate airflow to and from a patient; and
a multifunctional lumen disposed in a wall of the tubular body and configured to interchangeably receive a plurality of specialty devices, wherein a diameter of the multifunctional lumen is at least approximately 1.0 mm.

17. The multifunctional tracheal tube system of claim 16, wherein the diameter of the multifunctional lumen is at least approximately 2 mm.

18. The multifunctional tracheal tube system of claim 16, wherein the diameter of the multifunctional lumen is at least approximately 3 mm.

19. The multifunctional tracheal tube system of claim 16, wherein the plurality of specialty devices comprises an imaging device, a drug delivery device, a pressure sensor, a chemical sensor, and a position sensor.

20. The multifunctional tracheal tube system of claim 16, wherein the multifunctional lumen comprises a lubrication coating configured to facilitate insertion and/or removal of the one of the plurality of specialty devices.

21. The multifunctional tracheal tube system of claim 16, wherein the multifunctional lumen terminates in a self-sealing port configured to encircle the one of the plurality of specialty medical devices when inserted into the multifunctional lumen and to seal upon removal of the specialty medical device to maintain a separation between an interior of the multifunctional lumen and a surrounding environment.

22. A method, comprising:
intubating a patient with a tracheal tube comprising a main lumen and at least one multifunctional lumen disposed in a wall of the tracheal tube;
inserting a first specialty device into the multifunctional lumen without extubating the patient and while maintaining a breathing circuit between the patient and a ventilator;
removing the first device from the multifunctional lumen; and
inserting a second specialty device into the multifunctional lumen without extubating the patient and while maintaining a breathing circuit between the patient and the ventilator.

23. The method of claim 22, further comprising extubating the tracheal tube from the patient.

24. The method of claim 22, wherein the tracheal tube comprises a second multifunctional lumen configured to interchangeably receive at least two specialty devices comprising an imaging device, a drug delivery device, a position sensor, a chemical sensor, and a pressure sensor.

26. The method of claim 24, further comprising inserting at least one of the imaging device, the drug delivery device, the position sensor, and the pressure sensor into the second multifunctional lumen without extubating the patient and while maintaining the breathing circuit between the patient and the ventilator.

27. A multifunctional tracheal tube system, comprising:
a multifunctional tracheal tube comprising a tubular body, a ventilation lumen disposed in the tubular body and configured to facilitate airflow to and from a patient, a first lumen disposed in a wall of the tubular body, and a second lumen disposed in the wall of the tubular body;
at least two specialty devices; and
wherein the first lumen is configured to receive a first specialty device therethrough, and the second lumen is configured to receive a second specialty device therethrough.

28. The multifunctional tracheal tube system of claim 27, wherein the specialty devices comprise an imaging device, a drug delivery device, a position sensor, a chemical sensor, and a pressure sensor.

29. The multifunctional tracheal tube system of claim 27, wherein the tracheal tube includes a first marking indicating a first location of the first lumen and a second marking indicating a second location of the second lumen.

30. The multifunctional tracheal tube system of claim 27, wherein the first lumen and the second lumen are differently sized.

31. The multifunctional tracheal tube system of claim 27, wherein the first lumen and the second lumen terminate at different locations along the tubular body.

32. The multifunctional tracheal tube system of claim 31, wherein the locations of termination of the first lumen and the second lumen are provided based upon the purpose of the first specialty device and the second specialty device, respectively.
